(19) United States
(12) Patent Application Publication
(10) Pub. No.: US 2022/0168421 A1
(43) Pub. Date: Jun. 2, 2022

(54) PHOTOTHERMAL THERAPY PROMOTES TUMOR INFILTRATION AND ANTITUMOR ACTIVITY OF CART T CELLS

(71) Applicant: NORTH CAROLINA STATE UNIVERSITY, Raleigh, NC (US)

(72) Inventors: Zhen GU, Los Angeles, CA (US); Qian CHEN, Raleigh, NC (US)

(21) Appl. No.: 17/437,173

(22) PCT Filed: Mar. 9, 2020
(86) PCT No.: PCT/US2020/021664

§ 371 Date: Sep. 8, 2021

Related U.S. Application Data
(60) Provisional application No. 62/816,002, filed on Mar. 8, 2019.

(51) Int. Cl. A61K 41/00; A61K 35/17

(52) U.S. Cl. CPC A61K 41/0057; A61K 35/17

(57) ABSTRACT

Disclosed are engineered particles comprising a photosensitizer and methods for treating cancer comprising administering the engineered particles and tumor-specific T cells to a subject, wherein the photosensitizer is stimulated by light comprising a wavelength that excites the photosensitizer.

This application claims the benefit of U.S. Provisional Application No. 62/816,002, filed on Mar. 8, 2019, which is incorporated herein by reference in its entirety.

### I. BACKGROUND

T cells genetically engineered with chimeric antigen receptor (CAR) are radically innovative and sophisticated methods for cancer treatment. CARs are usually composed of the antigen-targeting region of a monoclonal antibody fused to the signaling molecules of the T cell receptor and costimulatory molecules. CD19-specific CAR T cells have been approved by Food and Drug Administration (FDA) to treat B cell malignancies. However, the efficacy of CAR T cells against solid tumors remains modest mostly because of the inefficient infiltration of CAR T cells into the tumor and the abundant presence of immunosuppressive cells. To exploit their effector function CAR T cells must engage chemotactic signals to traffic and accumulate into the tumor. The physical barriers represented by the extracellular matrix and stroma, together with the abnormal tumor vasculature and high interstitial fluid pressure (IFP) prevent the adequate infiltration of CAR T cells. The development of strategies to promote the infiltration of CAR T cells in solid tumors has become one of the major themes in the field. What are needed are new CAR T cell therapies that avoid the physical limitations imposed by the tumor and tumor vasculature.

### II. SUMMARY

Disclosed are compositions and methods related to an engineered particle comprising a photosensitizer which can be used as to recruit tumor-specific T cells to tumor sites.

Also disclosed herein are engineered particles of any preceding aspect, wherein the photosensitizer is encapsulated in the engineered particle; wherein the photosensitizer comprises a near-infrared (NIR) dye; and wherein the engineered particle comprises poly(lactic-co-glycolic) acid.

In one aspect, disclosed herein is a pharmaceutical composition comprising the engineered particle of any preceding aspect.

In one aspect, disclosed herein are methods of treating, inhibiting, reducing, decreasing, ameliorating and/or preventing a cancer and/or a metastasis in a subject comprising administering to the subject tumor-specific T cell population and effective amount of the engineered particle of any preceding aspect; and stimulating the engineered particle with light comprising a wavelength at which photosensitizer is excited.

In one aspect, disclosed herein are methods of treating, inhibiting, reducing, decreasing, ameliorating and/or preventing a cancer and/or a metastasis comprising administering to a subject in need thereof an effective amount of a tumor-specific T cell population and an engineered particle comprising a photosensitizer; and stimulating the engineered particle with light comprising a wavelength at which photosensitizer is excited; wherein the tumor-specific T cell population comprises CAR T, tumor infiltrating lymphocyte (TIL), effector T cell, memory T cell, effector memory RA T cell (TEMRA), or stem cell-like memory T cell.

Also disclosed herein are methods of treating, inhibiting, reducing, decreasing, ameliorating and/or preventing a cancer and/or a metastasis (including skin cancer, prostate cancer, lung cancer, breast cancer, pancreatic cancer, colon cancer, gastric cancer, bladder cancer, head and neck cancer, oral cancer, cholangiocarcinoma, ovarian cancer, cervical cancer, or esophageal cancer) in a subject comprising administering to the subject with a cancer the engineered particle of any preceding aspect. In one aspect, the subject is a mammal. In one aspect, the subject is a human.

In one aspect, disclosed herein are methods of treating, inhibiting, reducing, decreasing, ameliorating and/or preventing a cancer and/or a metastasis in a subject of any preceding aspect, wherein the engineered particles are administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, or once every 2, 3, 4, 5, 6 months.

Also disclosed herein are methods of treating, inhibiting, reducing, decreasing, ameliorating and/or preventing a cancer and/or a metastasis in a subject of any preceding aspect, wherein 1, 2, 3, 4, 5, 6, 7, 8, 9, or 10 doses of the engineered particles are administered to the subject; wherein the dose of the administered engineered particles is from about 1 mg/kg to about 100 mg/kg; and wherein the administering comprises intratumoral injection.

In one aspect, disclosed herein are methods of treating, inhibiting, reducing, decreasing, ameliorating and/or preventing a cancer and/or a metastasis in a subject of any preceding aspect, wherein the light comprises a NIR light. In one aspect, the NIR light comprises a wavelength of about 650 nm to about 1000 nm. In one aspect, the duration of stimulation is from 1 min to 60 min.

Also disclosed herein are methods of treating, inhibiting, reducing, decreasing, ameliorating and/or preventing a cancer and/or a metastasis in a subject of any preceding aspect, wherein comprising administering to the subject at least one anti-cancer therapeutic agent. In one aspect, the at least one anti-cancer therapeutic agent comprises an immune checkpoint blockade. In one aspect, the immune checkpoint blockade comprises an antibody targeting PD-1, PD-L1, PD-L2, or CTLA-4.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the effects of the mild heating of the tumor that causes enhanced infiltration and activation of adoptively transferred CAR.CSPG4⁺ T cells.

FIGS. 2A, 2B, 2C, 2D, 2E, 2F, 2G, and 2H show that photothermal therapy of the tumor promotes CAR T cell proliferation and cytokine release. FIG. 2Aa shows hydrodynamic diameter of PLGA-ICG nanoparticles measured by dynamic light scattering. Inset is the TEM image of PLGA-ICG (Scale bar, 200 nm). FIG. 2B show the UV-vis-NIR spectrum of PLGA-ICG, exhibiting high absorption in the near infrared region. FIGS. 2C and 2D show IR thermal images and temperature curves of PBS and PLGA-ICG under the 808-nm light irradiation for 5 min at the power density of 0.5 W/cm². Data are presented as mean±s.e.m. (n=3). FIG. 2E shows representative flow cytometry analysis of CAR.CSPG4⁺ T cells labeled with CFSE three days after the indicated treatments. FIG. 2F shows mean fluorescence intensity of CFSE, indicating T cell proliferation. Data are presented as mean±s.e.m. (n=4). FIG. 2G and FIG. 2H show detection of IL-2 and IFN-γ in the supernatant of CAR.CSPG4⁺ T cells, three days after the indicated treatments. Data are presented as mean±s.e.m. (n=4). Statistical significance was calculated via one-way ANOVA with a Tukey post-hoc test. P value: *P<0.05; ** P<0.01; ***P<0.001.

FIG. 3A and FIG. 3B show confocal fluorescence images of Calcein AM/PI (a) and flow cytometry analysis of annexin V/PI (b) co-stained WM115 cells incubated with PLGA-ICG one hour after being exposed to the 808 nm laser at different power densities. Scale bar, 50 μm.

FIG. 4 shows representative plot and CAR.CSPG4 expression on T cells after a week of culture.

FIGS. 5A, 5B, 5C, 5D, 5E, 5F, 5G, 5H, and 5I show how photothermal therapy of the tumor modifies the tumor microenvironment. FIG. 5A show shows IR thermal images of WM115-tumor-bearing mice injected with PLGA-ICG or PBS with the 808 nm laser irradiation (0.3 W/cm², 20 min). FIG. 5B shows changes of the tumor temperature measured by the IR thermal imaging FIG. 5C shows immunofluorescence imaging of tumors collected from mice 24 hours after photothermal therapy. Scale bar, 50 μm. FIG. 5D shows ultrasound imaging illustrating the blood perfusion of the WM115 tumors. Microbubbles injected intravenously were used as the ultrasound contrast agent. FIG. 5E shows representative hypoxia and HIF1-α immunofluorescence staining of the tumors after photothermal therapy (Scale bar, 50 μm). FIG. 5F shows representative flow cytometry plots and quantification of murine CD45⁺ cells infiltrating the tumor after photothermal therapy. Data are presented as mean±s.e.m. (n=10). FIG. 5G and FIG. 5Hh show representative flow cytometry plot and quantification of murine CD11c⁺ (5G) and CD11b⁺ (5H) cells gating on CD45⁺ cells. Data are presented as mean±s.e.m. (n=10). FIG. 5I shows quantification of chemokines in the tumor (n=10). Statistical significance was calculated via two-tailed Student's t-test. P value: *P<0.05; **P<0.01; ***P<0.001.

FIGS. 6A, 6B, 6C, 6D, 6E, 6F, and 6G show that photothermal ablation of the tumor increases the infiltration of adoptively transferred CAR T cells. FIG. 6A shows in vivo bioluminescence imaging of the CAR.CSPG4⁺ T cells. FIG. 6B shows quantification of CAR.CSPG4⁺ T cells detected in the tumor with or without photothermal ablation. Data are presented as mean±s.e.m. (n=3). FIG. 6C shows representative flow cytometry plots of CAR.CSPG4⁺ T cells infiltrating the tumor. FIGS. 6D, 6E, and 6F show absolute frequency of CD3⁺ (6D), CD4⁺ (6E) and CD8⁺ T cells (6F) within tumors. Data are presented as mean±s.e.m. (n=4). FIG. 6G shows representative immunofluorescence of tumors showing CD4⁺ and CD8⁺ CAR T cells infiltrating the tumor. Scale bar 50 μm. Statistical significance was calculated via two-tailed Student's t-test. P value: *P<0.05; **P<0.01; ***P<0.001.

FIGS. 7A, 7B, 7C, 7D, 7E, and 7F show that combined photothermal ablation and adoptive transfer of CAR T cells inhibits the growth of the human melanoma WM115 in vivo. FIG. 7A shows representative bioluminescence of the WM115 tumors (CAR=4). FIG. 7B and FIG. 7C show individual (7B) and average (7C) bioluminescence kinetics. Day 0 indicate the day in which treatment was initiated. Data are presented as mean±s.e.m. (n=6). FIG. 7D shows murine IL-6 levels detected in the tumors 7 days after the indicated treatments. Data are presented as mean±s.e.m. (n=8). FIG. 7E and FIG. 7F show human IL-2 and IFN-γ levels detected in the tumor 7 days after the indicated treatments. Data are presented as mean±s.e.m. (n=8). Statistical significance was calculated via one-way ANOVA with a Tukey post-hoc test. P value: *P<0.05; **P<0.01; ***P<0.001

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “decrease” can refer to any change that results in a smaller gene expression, protein expression, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

A “composition” is intended to include a combination of active agent and another compound or composition, inert (for example, a detectable agent or label) or active, such as an adjuvant.

The term “carrier” or “pharmaceutically acceptable carrier” means a carrier or excipient that is useful in preparing a pharmaceutical or therapeutic composition that is generally safe and non-toxic, and includes a carrier that is acceptable for veterinary and/or human pharmaceutical or therapeutic use. As used herein, the terms “carrier” or “pharmaceutically acceptable carrier” encompasses can include phosphate buffered saline solution, water, emulsions (such as an oil/water or water/oil emulsion) and/or various types of wetting agents. As used herein, the term “carrier” encompasses any excipient, diluent, filler, salt, buffer, stabilizer, solubilizer, lipid, stabilizer, or other material well known in the art for use in pharmaceutical formulations and as described further below.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS AND METHODS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular engineered particle is disclosed and discussed and a number of modifications that can be made to a number of molecules including the engineered particle are discussed, specifically contemplated is each and every combination and permutation of engineered particle and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed are compositions and methods related to an engineered particle comprising a photosensitizer which can be used as to recruit tumor-specific T cells to tumor sites. In one aspect, the photosensitizer is encapsulated in the engineered particle.

To facilitate these functions, the engineered particle can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers.

In one aspect, the engineered particle can comprise a biocompatible polymer (such as, for example, methacrylated hyaluronic acid (m-HA)). In one aspect, biocompatible polymer can be crosslinked. Such polymers can also serve to slowly release the adipose browning agent and/or fat modulating agent into tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the engineered particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like. In one aspect, the core polymer can be flanked by polypeptide blocks.

Examples of diblock copolymers that can be used in the micelles disclosed herein comprise a polymer such as, example, polyethylene glycol (PEG), polyvinyl acetate, polyvinyl alcohol (PVA), polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA)

The photosensitizer are compounds or molecules that luminesce. Typically, photosensitizers absorb electromagnetic energy at one wavelength and emit electromagnetic energy at a second wavelength. Representative photosensitizers include, but are not limited to, 1,5 IAEDANS; 1,8-ANS; 4-Methylumbelliferone; 5-carboxy-2,7-dichlorofluorescein; 7-dimethylaminocoumarin-3-carboxylic acid; 5-Carboxyfluorescein (5-FAM); 5-Carboxynapthofluorescein; 5-Carboxytetramethylrhodamine (5-TAMRA); 5-Hydroxy Tryptamine (5-HAT); 5-carboxy-X-rhodamine (5-ROX); 6-carboxy-X-rhodamine (6-ROX); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 405™; Alexa Fluor 430™; Alexa Fluor 488™; Alexa Fluor 500™; Alexa Fluor 514™; Alexa Fluor 532™; Alexa Fluor 546™; Alexa Fluor 555™; Alexa Fluor 568™; Alexa Fluor 594™; Alexa Fluor 610™; Alexa Fluor 633™; Alexa Fluor 647™; Alexa Fluor 660™; Alexa Fluor 680™; Alexa Fluor 700™; Alexa Fluor 750™; Alexa Fluor 790™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; cinnamic acid; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; red cyanine dyes, Cy5/Alexa 647, cyclic AMP Fluorosensor (FiCRhR); Dabcyl; Dabsyl chloride; Dansyl; Dansyl Amine; Dansyl Cadaverine; Dansyl Chloride; Dansyl DHPE; Dansyl fluoride; 4′,6-diamidino-2-phenylindole (DAPI); Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; Dronpa; bsDronpa; DsRed; DTAF; DY-630-NHS; DY-635-NHS; EBFP; ECFP; EGFP; ELF 97; EOS, Eosin; Erythrosin; Erythrosin ITC; Ethidium Bromide; Ethidium homodimer-1 (EthD-1); Euchrysin; EukoLight; Europium (111) chloride; enhanced yellow fluorescent protein (EYFP); Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; fluorescein carboxylic acid; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indocyanine Green; Indodicarbocyanine (DiD); Indotricarbocyanine (DiR); Intrawhite Cf; Li-COr dyes; IR-800 CW; IR-800 Mal; IRdye800JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); nitrobenzodiazolamine (NBD); NBD Amine; Nile Blue; Nile Red; NIR641, NIR664, NIT7000, and NIR782Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylcarboxyrhodamine; Tetraethylsulfohodamine; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; ZW-800; Sybr Green; Thiazole orange (interchelating dyes); semiconductor nanoparticles such as quantum dots; or caged photosensitizer (which can be activated with light or other electromagnetic energy

Photothermal therapy employs optical absorbing agents to “burn” tumor cells by generating heat under the near-infrared (NIR) light irradiation. Compared to traditional cancer therapies, photothermal therapy has unique advantages that include high selectivity, low systemic toxicity and non-therapeutic resistance. When injected intratumorally, the engineered particles comprising a photosensitizer can promote direct tumor cell killing, partial disruption of the extracellular matrix, decrease of the IFP and increase of the blood perfusion. Hyperthermia destructs cancer cells and causes the inflammation in the tumor, which greatly enhance recruitment and activation of immune cells, including tumor specific T cells, in the tumor site, which significantly improves the cancer treatment efficacy. During treatment, a commercial NIR optical imager uses an LED, white light or a laser light source sending incident light into the patient's tissues that includes light from 650-790 nm. The NIR dye absorbs some of that light and emits further fluorescent light at 800-840 nm, preferably at >800 nm. Unlike the visible light spectrum (400-650 nm), in the NIR region, light scattering decreases and photo absorption by hemoglobin and water diminishes, leading to deeper tissue penetration of light. Furthermore, tissue auto-fluorescence is low in the NIR spectra, which allows for a high signal to noise ratio. There is a range of small molecule organic photosensitizers with excitation and emission spectra in the NIR region. Some, such as indocyanine green (ICG) and cyanine derivatives Cy5.5 and Cy7, have been used in imaging for a relatively long time. Modern photosensitizers are developed by various biotechnology companies and include: Li-COr dyes; IR-800 CW; IR-800 Mal; Alexa dyes; IR Dye dyes; VivoTag dyes and HylitePlus dyes. In addition to dyes used to emit in the near infra-red spectrum, dyes that emit above 780 nm and can extend into the near infrared II (NIR-II) spectrum from 1000 nm to 1700 nm. Preferably, the dye emits fluorescent light from about 800 nm to about 1700 nm. An example of a detectable label that emits between 780 nm and 1700 nm include dicyanine dye. Dicyanine dyes that are useful in this invention include IRdye800, AlexaFluor 790, ZW-800, Indocyanine Green, and the like.

In one aspect, disclosed herein is a pharmaceutical composition comprising any of the engineered particle disclosed herein.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, intratumorally, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHODS OF TREATING, INHIBITING, REDUCING, AMELIORATING, DECREASING, AND/OR PREVENTING A CANCER AND/OR METASTASIS

It is understood and herein contemplated that the disclosed engineered particles can be used to apply photothermal therapy to ‘burn’ tumor cells by effectively generating heat under the near-infrared (NIR) light irradiation through the use of optical absorbing agents. Additionally, the disclosed engineered particles can enhance the infiltration of T cells to a tumor site (including, but not limited to adoptively transferred T cells) thereby treating a cancer. Accordingly, the disclosed compositions can be used to treat, inhibit, reduce, decrease, ameliorate and/or prevent any disease where uncontrolled cellular proliferation occurs such as cancers and metastasis.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. In one aspect, disclosed herein are methods of treating, preventing, inhibiting or reducing a cancer or metastasis in a subject comprising administering to the subject an effective amount of the engineered particles disclosed herein; and stimulating the engineered particle with light comprising a wavelength at which photosensitizer is excited.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

As noted above, the photosensitizer of the disclosed particles can enhance the infiltration of T cells to a tumor site by causing photothermal ablation to the tumor microenvironment upon exposure to the appropriate wavelength of light given a particular photosensitizer. Representative photosensitizers that can be used in the disclosed methods include, but are not limited to, 1,5 IAEDANS; 1,8-ANS; 4-Methylumbelliferone; 5-carboxy-2,7-dichlorofluorescein; 7-dimethylaminocoumarin-3-carboxylic acid; 5-Carboxyfluorescein (5-FAM); 5-Carboxynapthofluorescein; 5-Carboxytetramethylrhodamine (5-TAMRA); 5-Hydroxy Tryptamine (5-HAT); 5-carboxy-X-rhodamine (5-ROX); 6-carboxy-X-rhodamine (6-ROX); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 405™; Alexa Fluor 430™; Alexa Fluor 488™; Alexa Fluor 500™; Alexa Fluor 514™; Alexa Fluor 532™; Alexa Fluor 546™; Alexa Fluor 555™; Alexa Fluor 568™; Alexa Fluor 594™; Alexa Fluor 610™; Alexa Fluor 633™; Alexa Fluor 647™; Alexa Fluor 660™; Alexa Fluor 680™; Alexa Fluor 700™; Alexa Fluor 750™; Alexa Fluor 790™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; cinnamic acid; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; red cyanine dyes, Cy5/Alexa 647, cyclic AMP Fluorosensor (FiCRhR); Dabcyl; Dabsyl chloride; Dansyl; Dansyl Amine; Dansyl Cadaverine; Dansyl Chloride; Dansyl DHPE; Dansyl fluoride; 4′,6-diamidino-2-phenylindole (DAPI); Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; Dronpa; bsDronpa; DsRed; DTAF; DY-630-NHS; DY-635-NHS; EBFP; ECFP; EGFP; ELF 97; EOS, Eosin; Erythrosin; Erythrosin ITC; Ethidium Bromide; Ethidium homodimer-1 (EthD-1); Euchrysin; EukoLight; Europium (111) chloride; enhanced yellow fluorescent protein (EYFP); Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; fluorescein carboxylic acid; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indocyanine Green; Indodicarbocyanine (DiD); Indotricarbocyanine (DiR); Intrawhite Cf; Li-COr dyes; IR-800 CW; IR-800 Mal; IRdye800JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); nitrobenzodiazolamine (NBD); NBD Amine; Nile Blue; Nile Red; NIR641, NIR664, NIT7000, and NIR782Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylcarboxyrhodamine; Tetraethylsulfohodamine; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; ZW-800; Sybr Green; Thiazole orange (interchelating dyes); semiconductor nanoparticles such as quantum dots; or caged photosnsitizer (which can be activated with light or other electromagnetic energy source), or a combination thereof.

Typically photosensitizers absorb electromagnetic energy at one wavelength and emit electromagnetic energy at a second wavelength. Photosensitizers emit energy, including thermal energy, throughout the visible spectrum as well as the near-infrared (NIR) region (650 nm-900 nm). Unlike the visible light spectrum (400-650 nm), in the NIR region, light scattering decreases and photo absorption by hemoglobin and water diminishes, leading to deeper tissue penetration of light. Furthermore, tissue auto-fluorescence is low in the NIR spectra, which allows for a high signal to noise ratio. There is a range of small molecule organic photosensitizers with excitation and emission spectra in the NIR region. Some, such as indocyanine green (ICG) and cyanine derivatives Cy5.5 and Cy7, have been used in imaging for a relatively long time. Modern photosensitizers are developed by various biotechnology companies and include: Li-COr dyes; IR-800 CW; IR-800 Mal; Alexa dyes; IRDye dyes; VivoTag dyes and HylitePlus dyes. In some aspects, the photosensitizer can be excited and/or emit into the near infrared II (NIR-II) spectrum from 1000 nm to 1700 nm. Preferably, the dye emits fluorescent light from about 800 nm to about 1700 nm. An example of a detectable labels that emits between 780 nm and 1700 nm include dicyanine dye. Dicyanine dyes that are useful in this invention include IRdye800, AlexaFluor 790, ZW-800, Indocyanine Green, and the like. Accordingly, in one aspect, disclosed herein is are methods of treating, preventing, inhibiting or reducing a cancer or metastasis in a subject in a, wherein the light comprises a NIR light. In one aspect, the NIR light comprises a wavelength of about 650 nm to about 1000 nm. In one aspect, the duration of stimulation is from 1 min to 60 min. Thus, disclosed herein is a methods of treating, preventing, inhibiting or reducing a cancer or metastasis (including skin cancer, prostate cancer, lung cancer, breast cancer, pancreatic cancer, colon cancer, gastric cancer, bladder cancer, head and neck cancer, oral cancer, cholangiocarcinoma, ovarian cancer, cervical cancer, or esophageal cancer) in a subject comprising administering to the subject with a cancer any of the engineered particles disclosed herein and exposing the subject to light that will cause the photosensitizer to emit thermal energy.

As noted above, any of the engineered particles disclosed herein can be used as part of a method of treating, preventing, inhibiting, or reducing a cancer or metastasis by utilizing the photothermal property of the photosensitizer on the engineered particle to enhance immune cell infiltration of the tumor site upon exposure to light. Thus, the methods can further comprise the administration of T cells. Accordingly, in one aspect, disclosed herein are methods of treating, inhibiting, reducing, decreasing, ameliorating and/or preventing a cancer and/or a metastasis in a subject comprising administering to the subject tumor-specific T cell population and effective amount of the engineered particle of any preceding aspect; and stimulating the engineered particle with light comprising a wavelength at which photosensitizer is excited.

The T-cells of the subject can be obtained from the subject by any means appropriate to recover at least some live T-cells. For example, the T-cells can be obtained from a biological sample of the subject. The biological sample can be any T-cell-containing biological sample, for example, blood, plasma, lymph, tissue, tumor biopsy, and the like. The biological sample can be obtained by standard medical, clinical, and/or phlebotomy techniques, and the biological sample can be further processed as required (e.g., purification, culture, storage). In one aspect, the T cells can be endogenous T cells recruited to the tumor microenvironment due to the thermal ablation caused by the engineered particle when exposed to light.

T cells used in the disclosed methods can be engineered prior to administration. T cells genetically engineered with chimeric antigen receptor (CAR) are radically innovative and sophisticated methods for cancer treatment. CARs are usually composed of the antigen-targeting region of a monoclonal antibody fused to the signaling molecules of the T cell receptor and costimulatory molecules. The unique strength of CAR T is its ability to target intracellular and extracellular antigens of interest. Disclosed herein are methods for expanding CAR T cells for use in cancer therapy. The disclosed methods comprise providing a CAR T cell comprising T cell receptor specific to the antigen, but not limited to, chondroitin sulfate proteoglycan-4 (CSPG4), which is overexpressed by melanoma and glioblastoma but with limited distribution in normal tissues. An array of T-cell types are compatible with the herein disclosed methods, for example effector T-cells, helper T-cells, cytotoxic T-cells, memory T-cells, regulatory T-cells, gamma-delta T-cells, TIL, engineered T cells, CAR T cells, TEMRA, stem cell-like memory T cell etc. In some embodiments, the T-cells comprise CD4+ T-cells, CD8+ T-cells, or combinations thereof. CD8+ T-cells are also referred to as cytotoxic T-cells and can function to kill specifically recognized cells (e.g., tumor cells). In some embodiments, the cells are isolated or purified. In one aspect, it is understood and herein contemplated that source and recipient of the administered T cells can be the same or different subjects. It is further understood and herein contemplated that the administered T cells can be modified (for example a CAR T cell) prior to being administered to the recipient subject. Thus, in one aspect, disclosed herein are methods of treating, preventing, inhibiting or reducing a cancer or metastasis in a subject comprising administering to the subject tumor-specific T cell population (such as, for example a CAR T cell or TIL population) and an effective amount of the engineered particles disclosed herein; and stimulating the engineered particle with light comprising a wavelength at which photosensitizer is excited. Thus, in one aspect, disclosed herein are methods of treating, inhibiting, reducing, decreasing, ameliorating and/or preventing a cancer and/or a metastasis comprising administering to a subject in need thereof an effective amount of a tumor-specific T cell population and an engineered particle comprising a photosensitizer; and stimulating the engineered particle with light comprising a wavelength at which photosensitizer is excited; wherein the tumor-specific T cell population comprises CAR T, tumor infiltrating lymphocyte (TIL), effector T cell, memory T cell, effector memory RA T cell (TEMRA), or stem cell-like memory T cell.

In one aspect, the subject is a mammal. In one aspect, the subject is a human.

The disclosed engineered particles can be administered either intratumorally or systemically (such as, for example, by intravenous injection). In one aspect, disclosed herein are methods of treating, inhibiting, reducing, decreasing, ameliorating and/or preventing a cancer and/or a metastasis comprising administering to a subject in need thereof an effective amount of and an engineered particle comprising a photosensitizer; wherein the engineered particle is administered directly to the tumor at the tumor site (intratumorally) or systemically (such as, for example, by intravenous injection). It is also understood and herein contemplated that when administered as part of the disclosed methods of treating, inhibiting, reducing, decreasing, ameliorating and/or preventing a cancer and/or a metastasis, the tumor-specific T cell population can also be administered intratumorally or systemically. It is understood and herein contemplated that the T cells and the engineered particles can both be administered intratumorally or systemically. Alternatively, either the T cells or the engineered particles can be administered intratumorally while the other is administered systemically. In one aspect, disclosed herein are methods of treating, inhibiting, reducing, decreasing, ameliorating and/or preventing a cancer and/or a metastasis comprising administering to a subject in need thereof an effective amount of a tumor-specific T cell population and an engineered particle comprising a photosensitizer; wherein the engineered particles are administered intratumorally and the T cells are administered systemically (such as, for example, by intravenous injection).

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

In one aspect, disclosed herein is a method of treating, preventing, inhibiting, or reducing a cancer or metastasis comprising administering to a subject the engineered particles at any frequency appropriate for the treatment of the particular cancer in the subject. For example, engineered particles can be administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months.

Also disclosed herein is a method of treating a cancer in a subject, wherein 1, 2, 3, 4, 5, 6, 7, 8, 9, or 10 doses of the engineered particles are administered to the subject.

It is understood and herein contemplated that the engineered particle and tumor-specific T cells are administered to a subject separately (concurrent or sequential administration). For example, the engineered particle can be administered to the site of the tumor in the subject at least 1, 2, 3, 4, 5, 10, 15, 20, 25, 30, 35, 40, 45, 50, 55 minutes, 1, 2, 3, 4, 5, 6, 7, 8, 9, 10, 11, 12, 18, 24, 36, 48, or 72 hours before the tumor-specific T cell. Similarly, the engineered particle can be administered to the site of the tumor in the subject at least 1, 2, 3, 4, 5, 10, 15, 20, 25, 30, 35, 40, 45, 50, 55 minutes, 1, 2, 3, 4, 5, 6, 7, 8, 9, 10, 11, 12, 18, 24, 36, 48, or 72 hours after the tumor-specific T cell.

In one aspect, the amount of the engineered particles disclosed herein which are administered to the subject for use in the disclosed methods can comprise any amount appropriate for the treatment of the subject for the particular cancer as determined by a physician. For example, the amount of the engineered particles can be from about 10 mg/kg to about 100 mg/kg. For example, the amount of the pharmaceutical compositions, engineered particles, and/or engineered particles administered can be at least 10 mg/k, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. Accordingly, in one aspect, disclosed herein are methods of treating a cancer in a subject, wherein the dose of the administered engineered particles is from about 10 mg/kg to about 100 mg/kg.

Also disclosed herein are methods of treating, preventing, inhibiting or reducing a cancer or metastasis comprising administering to the subject at least one anti-cancer therapeutic agent, including, but not limited to, Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

Also noted herein, disclosed herein are methods of treating, preventing, inhibiting or reducing a cancer or metastasis comprising administering to the subject at least one anti-cancer therapeutic agent. In one aspect, the at least one anti-cancer therapeutic agent comprises an antibody targeting immune checkpoint blockade. In one aspect, the blockade inhibitor that can be used in the disclosed methods can be any inhibitor of an immune checkpoint such as for example, a PD-1/PD-L1 blockade inhibitor, a CTLA-4/B7-1/2 blockade inhibitor (such as for example, Ipilimumab), and CD47/Signal Regulator Protein alpha (SIRPα) blockade inhibitor (such as for example, Hu5F9-G4, CV1, B6H12, 2D3, CC-90002, and/or TTI-621). Examples, of PD-1/PD-L1 blockade inhibitors for use in the disclosed engineered particless can include any PD-1/PD-L1 blockade inhibitor known in the art, including, but not limited to nivolumab, pembrolizumab, pidilizumab, atezolizumab, avelumab, durvalumab, and BMS-936559). It is understood and herein contemplated that the engineered particles can be designed to incorporate 1, 2, 3, 4, 5, 6, 7, 8, 9, 10, 11, 12, 13, 14, or 15 blockade inhibitors simultaneously.

The methods can include administering engineered particles and T-cells of the subject formulated with a pharmaceutically acceptable carrier and/or as a medicament. Suitable carriers include, but are not limited to, salts, diluents, binders, fillers, solubilizers, disintegrants, preservatives, sorbents, and other components.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for.

### 1. Example 1: Photothermal Therapy Promotes Tumor Infiltration and Antitumor Activity of CAR T Cells

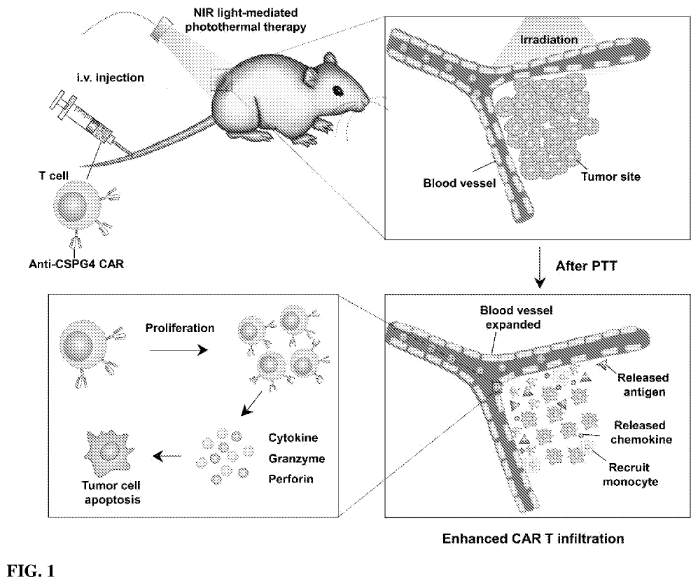

Photothermal therapy employs optical absorbing agents to ‘burn’ tumor cells by effectively generating heat under the near-infrared (NIR) light irradiation. Compared to traditional cancer therapies, photothermal therapy has unique advantages that include high selectivity, low systemic toxicity and non-therapeutic resistance. As shown in FIG. 1, poly(lactic-co-glycolic) acid (PLGA) nanoparticle is loaded with indocyanine green (ICG), an NIR dye a as photothermal agent. When injected intratumorally, these PLGA-ICG nanoparticles can promote direct tumor cell killing, partial disruption of the extracellular matrix, decrease of the IFP and increase of the blood perfusion. Moreover, the destruction of cancer cells by hyperthermia causes the inflammation in the tumor, which can enhance recruitment of immune cells and secrete a variety of chemokines and cytokines. Working together with the released tumor-specific antigens after photothermal ablation, this therapy greatly enhances the infiltration and activation of CAR T cells in the tumor site, for significantly improving the treatment efficacy.

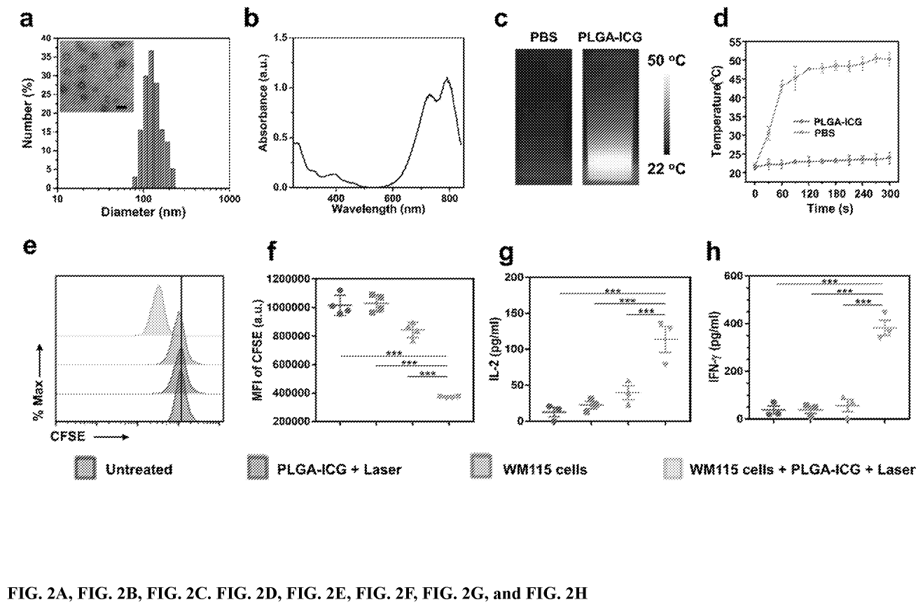

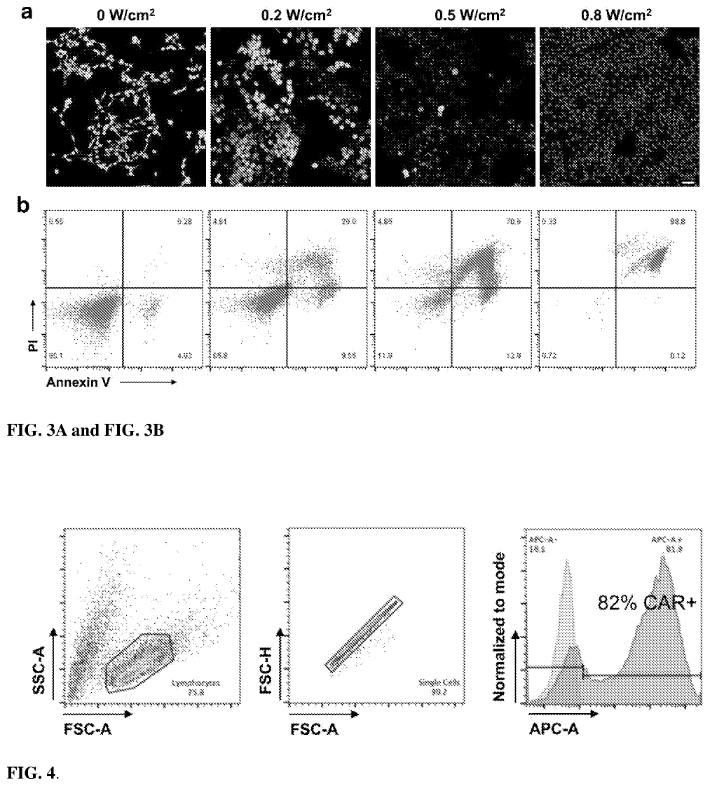

PLGA, a polymer applied in US FDA approved formulations, was used to encapsulate ICG, an NIR dye for photothermal therapy, via an oil-in-water (o/w) emulsion method. Monodispersed PLGA-ICG nanoparticles (˜100 nm) with spherical shape were obtained, as revealed by the transmission electron microscope (TEM) imaging and dynamic light scattering (DLS) (FIG. 2a). PLGA-ICG nanoparticles exhibited a characteristic absorption peak of ICG at ˜780 nm, which is ideal for effective photothermal therapy (FIG. 2b). Temperature elevation was observed when the PLGA-ICG solution was irradiated by the 808 nm laser (0.5 W/cm² for 5 min), while pure phosphate buffered saline (PBS) solution showed modest temperature increase under the same laser irradiation (FIGS. 2c and d). Confocal fluorescent images of propidium iodide (PI) and Calcine AM co-stained cells and the flow cytometry results of cell stained with PI and Annexin V further confirmed the effective photothermal ablation of cancer cells (FIG. 3).

Next, the effect of ex vivo photothermal exposure of the tumor cells on CAR T cell function was shown. CAR T cells specific to the antigen chondroitin sulfate proteoglycan-4 (CSPG4) were used to target this antigen overexpressed melanoma and glioblastoma; while limited distribution is observed in normal tissues^{[12]}. T lymphocytes obtained from the healthy donors were engineered to express the CSPG4.CAR (FIG. 4). The proliferation of CAR.CSPG4⁺ T cells was evaluated using the carboxyfluorescein diacetate succinimidyl ester (CFSE)-based assay. The melanoma cell line WM115 that express CSPG4 with or without photothermal exposure was plated in the upper chamber of a transwell with the pore size ˜1 μm and CAR.CSPG4⁺ T cells were plated in the lower chamber. After three days of culture, CSPG4.CAR T cells vigorously proliferated after coculture with WM115 cells upon photothermal ablation, showing that the CSPG4 protein released form WM115 cells after photothermal therapy engage and stimulate CSPG4-specific CAR T cells (FIGS. 2d and e). The activation of CSPG4.CAR T cells in response to WM115 cells after photothermal ablation was further indicated by the increased production of interleukin-2 (IL-2) and interferon-γ (IFN-γ) (FIGS. 2f and g).

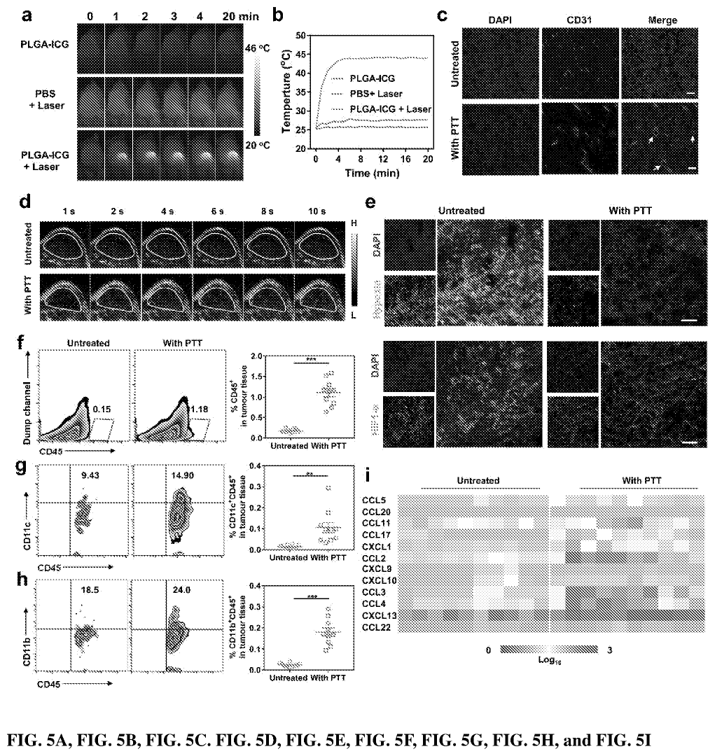

To validate whether the photothermal therapy can enhance the antitumor effects of CAR T cells in solid tumors, the effects of mild photothermal ablation in NSG mice bearing the human melanoma WM115 tumor subcutaneously were characterized. The PLGA-ICG nanoparticles were injected, following with irradiation of the tumor with the 808 nm laser at the power density of 0.3 W/cm². The temperature of the tumor injected with PLGA-ICG increased to ˜44° C. within 2 minutes as monitored by an infrared thermal camera (FIGS. 5a and b). After the photothermal treatment, the morphology of the tumor vasculature appeared dilated with reduced IFP as compared to control tumors (FIG. 5c). Twenty four hours after photothermal ablation ultrasound imaging with a microbubble contrast agent showed higher signals further indicating the reduction of the IFP within the tumor (FIG. 5d). Moreover, the signals of the hypoxia probe pimonidazole and the hypoxia-inducible factor (HIF)-1α were also decreased, showing enhanced oxygenation (FIG. 5e). In addition, photothermal ablation caused the intratumor increase of murine monocytes (CD45⁺CD11b⁺) and dendritic cells (CD45⁺CD11c⁺) (FIG. 5f-h) as well as murine chemokines such as chemokine ligand 5 (CCL5), CCL 11, chemokine (C-X-C motif) ligand 1 (CXCL1), CCL 2, CCL 3, and CCL 4 (FIG. 5i).

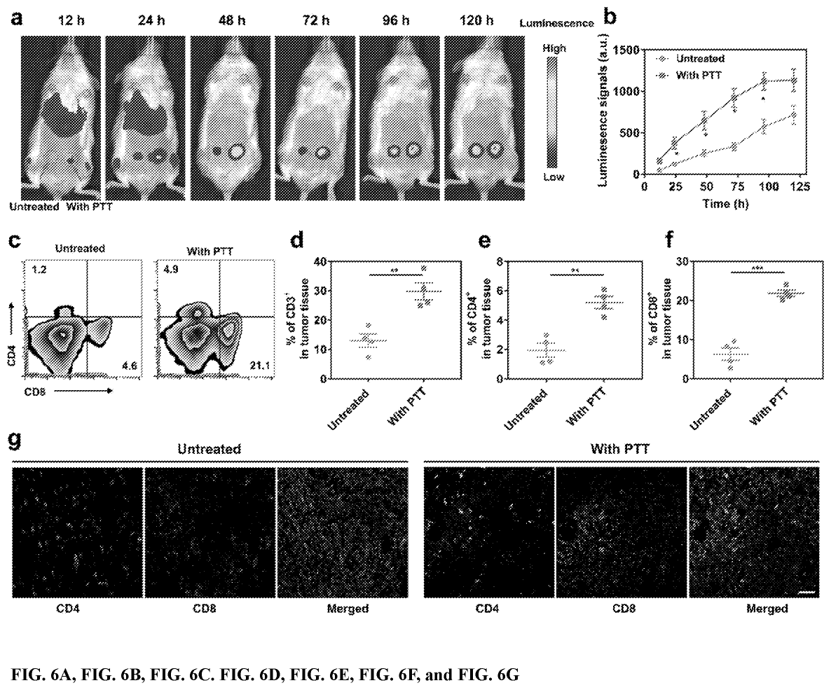

Next, a xenograft model of melanoma was established by inoculating WM115 tumor cells into both flanks of NSG mice. After three weeks, the tumors in the right flank were intratumorally injected with PLGA-ICG and irradiated with the 808 nm laser. Two hours later, 1×10⁷ CAR.CSPG4⁺ T cells or CAR.CD19⁺ T cells labeled with the firefly luciferase were intravenously injected. T cell biodistribution was monitored by an in vivo imaging (IVIS) at different time points after CAR T cell administration. Increased localization of CAR.CSPG4⁺ T cells was observed in tumors that received photothermal therapy as compared to the contralateral tumors (FIGS. 6a and b). Flow cytometry (FIG. 6c-f) and immunofluorescence imaging (FIG. 6g) confirmed the accumulation of CAR.CSPG4⁺ T cells in the tumors treated with photothermal therapy.

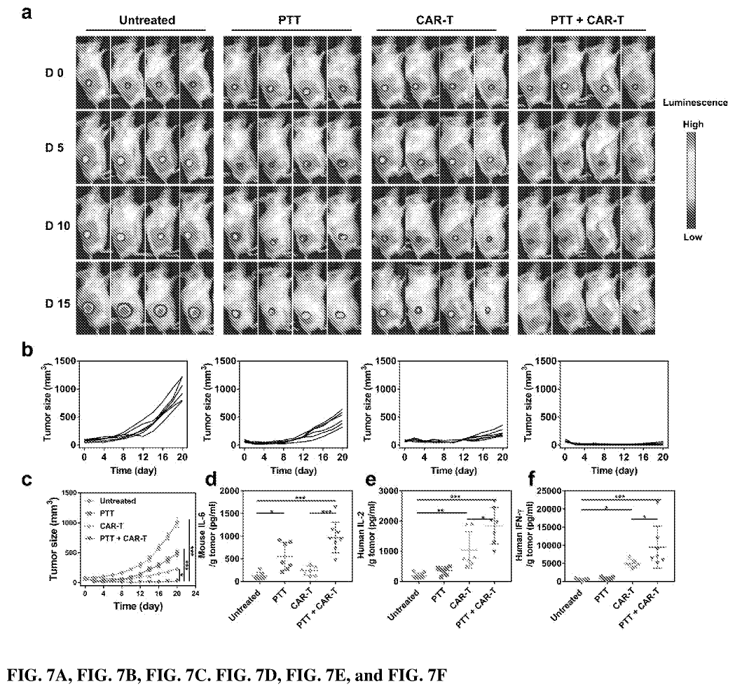

To assess the antitumor activity of combining photothermal therapy and CAR.CSPG4⁺ T cells, the WM115 human melanoma tumor cell line was labeled with firefly luciferase. WM115 tumor bearing mice were intratumorally injected with PLGA-ICG and irradiated with the 808 nm laser for 20 minutes at a power density of 0.3 W/cm². Two hours later, 1×10⁷ CAR.CSPG4⁺ T cells were intravenously injected into the mice. Tumor growth was monitored using both in vivo bioluminescence (FIG. 7a) and caliper measurement (FIGS. 7b and c). Photothermal therapy combined with CAR.CSPG4⁺ T cells significantly suppressed the tumor growth up to 20 days as compared with control groups. Two out of six mice receiving the combined treatment were macroscopically tumor free at the end of the experiment. In parallel experiments, cytokine levels in treated mice were measured. Murine IL-6 was increased after photothermal therapy (FIG. 7d). Moreover, human IL-2 and IFN-γ released by CAR T cells were also significantly increased especially in mice reeving the combined treatment (FIGS. 7e and f).

In summary, mild heating of the tumor triggers physicochemical and physiological changes of the tumor, resulting in increased infiltration and accumulation of CAR.CSPG4⁺ T cells. In addition to directly killing tumor cells, mild heating could also destruct partial tumor cells and extracellular matrix, thereby, reducing the compact degree of solid tumor, decreasing IFP and expanding blood vessels in the tumor. Moreover, tumor-associated antigens produced from the ablated tumor residues after photothermal therapy recruit endogenous immune cells and activate the immune system. Facilitated by the photothermal therapy, CAR.CSPG4⁺ T lymphocytes traffic to and accumulate at the tumor site, following the chemokine and antigen attraction. In NSG mice engrafted with the human melanoma WM115 tumors, effective therapeutic effect was achieved after mild heating of solid tumor (˜44° C.) and subsequently intravenous infusion of CAR.CSPG4⁺ T cells. Thus, this combination provides a promising platform to simply and safely increase the therapeutic index of CAR T cells in solid tumors. This platform can be further potentiated by tuning treatment variables including duration and frequency of the photothermal and by including targeted immunomodulatory therapeutics.

### 2. Example 2: Methods and Materials

All chemicals were obtained from Sigma-Aldrich and used without any purification. Human melanoma WM115 cells and WM115-luc cells were cultured in RPMI 1640 (HyClon) medium containing 10% heat inactivated fetal calf serum (F Invitrogen, Carlsbad, Calif.), 2 mmol/L GlutaMAX (Invitrogen), 200 IU/mL penicillin, and 200 mg/mL streptomycin (Invitrogen) in an incubator at 37° C. in 5% CO₂. The CD19-specific CAR T lymphocytes and CSPG4-specific CAR T lymphocytes also were generated in Dr. Gianpietro Dotti's lab at UNC^{[12b]}. CAR T cells were cultured and expanded in complete medium containing 45% RPMI 1640 and 45% Click's medium (Irvine Scientific) with 10% FCS (HyClone), 2 mmol/L GlutaMAX, 100 IU/mL penicillin, and 100 mg/mL streptomycin. Cells were fed twice a week with recombinant interleukin-7 (IL-7) (5 ng/mL; Pepro Tech Inc) and interleukin-15 (IL-15) (10 ng/mL; Pepro Tech Inc). Female NSG mice (6-10 weeks) were purchased from Jackson Lab. All mouse studies were carried out following the protocols approved by the Institutional Animal Care and Use Committee at the University of North Carolina at Chapel Hill and North Carolina State University and complied with all relevant ethical regulations.

a) Synthesis and Characterization of PLGA-ICG Nanoparticles.

PLGA-ICG nanoparticles were prepared using an o/w single-emulsion method^{[10]}. Briefly, photothermal agent ICG was dissolved in DMSO at 10 mg/ml, and then added to PLGA dichloromethane solution. The mixture was homogenized with 5% w/v PVA solution by Selecta Sonopuls for 10 min. Then, the emulsion was added to additional 5% w/v solution of PVA to evaporate the organic solvent. PLGA-ICG nanoparticles were obtained after centrifugation at 3,500 g for 20 min. The morphology of PLGA-ICG nanoparticles was characterized by TEM (JEOL 2000FX) and the size distribution was measured by the Zetasizer Nano-ZS (Malvern Instruments, UK). The absorbance spectrum was recorded by Nanodrop.

b) Cellular Experiments.

To study the proliferation the CAR.CSPG4⁺ T lymphocytes, CAR.CSPG4⁺ T lymphocytes (1×10⁶ cells) were stained with carboxyfluorescein succinimidyl ester (CFSE, 5 μM) according to the protocol of the Cell Trace™ CFSE Cell Proliferation Kit (Invitrogen). Then, CAR.CSPG4⁺ T lymphocytes were incubated with PLGA-ICG nanoparticle solution after photothermal ablation, WM115 cells or WM115 cells after photothermal ablation using a transwell system (400 nm) for 3 days in the incubator. The fluorescent intensity of CFSE was detected by flow cytometry to monitor the proliferation of T cells. Meanwhile, the medium supernatant of CAR.CSPG4⁺ T lymphocytes was collected and the different cytokines including interleukin 2 (IL-2) and interferon-γ (IFN-γ) were measured by an enzyme-linked immunosorbent assay (ELISA).

c) In Vivo Tumor Models and Treatment.

For the in vivo biodistribution of CAR.CSPG4⁺ T lymphocytes, 5×10⁶ WM115 human melanoma cells were subcutaneously injected into both sides of each mouse. After ˜20 days, when the tumor volume reached ˜100 mm³, PLGA-ICG nanoparticles were intratumorally injected into the right tumors and irradiated with an 808 nm laser at the power density of 0.3 W/cm² for 20 min. Two hours later, 1×10⁷ CAR.CSPG4⁺ T lymphocytes labeled with luciferase were intravenously injected into the mice. Different time points after intravenous injection of T cells, mice were imaged via an IVIS Spectrum Imaging System (Perkin Elmer Ltd) for 1 min to monitor the biodistribution of T cells.

For in vivo combination therapy, NSG mice bearing subcutaneous fLuc-WM115 tumors were divided into four groups (n=6 per group): (a) untreated; (b) intratumorally injected with PLGA-ICG nanoparticles and irradiated by the 808-nm laser (0.3 W/cm², 10 min); (c) intravenously injected with 1×10⁷ CAR.CSPG4⁺ T lymphocytes only; (d) intratumorally injected with PLGA-ICG nanoparticles and irradiated by the 808-nm laser (0.3 W/cm², 10 min), and then intravenously injected with 1×10⁷ CAR.CSPG4⁺ T lymphocytes. The changes of the temperature on the surface of tumor were monitored by an IR thermal camera. The tumor sizes were recorded by a digital caliper every 2 days and calculated according to the following formula: width²×length×0.5. The tumor also was monitored using an in vivo bioluminescence imaging system. Ten minutes after intraperitoneal injection of d-luciferin (Thermo Scientific™ Pierce™, 150 mg/kg) into each mouse, mice were imaged via an IVIS Spectrum Imaging System for 1 s.

d) Immunofluorescence Staining.

Tumors were collected from mice, fixed, and stained according to standard procedures. To study the changes after photothermal ablation, 24 h after photothermal ablation, the blood vessels were stained with anti-CD31 primary antibody (Abcam, cat. no. ab28364) and goat anti-rabbit IgG (H+L; Thermo Fisher Scientific, cat. no. A 11037). For tumor hypoxia study, 90 min before tumors were surgically excised from the mice, pimonidazole hydrochloride (60 mg/kg) (Hypoxyprobe-1 plus kit, Hypoxyprobe Inc) was intraperitoneally injected into the mice. Then, the tumor sections were incubated with mouse anti-pimonidazole antibody (Hypoxyprobe Inc.) or anti-HIF-1α antibody (Abcam, cat. no. ab ab16066) overnight, and then stained with goat anti-mouse IgG (H+L; Thermo Fisher Scientific, cat. no. 62-6511). For T cells detection, the tumor sections were labelled with primary antibodies: CD4 (Abcam, cat. no. ab133616) and CD8 (Abcam, cat. no. ab17147) overnight and then stained with fluorescently labelled secondary antibodies: goat anti-rabbit IgG (H+L; Thermo Fisher Scientific, cat. no. A16111) and goat anti-mouse IgG (H+L; Thermo Fisher Scientific, cat. no. M32017). Then, the slides were analyzed via the confocal imaging (Zeiss LSM 710).

e) Chemokine and Cytokine Detection.

The different chemokine concentrations in the tumor were measured by LEGENDplex mouse proinflammatory chemokine panel multiple assay (catalog no. 740007, BioLegend) according to the manufacturer's instructions. 24 h after photothermal ablation, the tumor tissue was harvested and then homogenized in cold PBS buffer in the presence of protease inhibitor. The supernatant was collected for detection. For detection of the local concentration of IL-2 and IFN-γ secreted by CAR.CSPG4⁺ T cells, 7 days after different treatments, the tumor tissues were harvested and then homogenized in cold PBS buffer in the presence of protease inhibitor for detection.

f) Flow Cytometry.

To detect the changes after photothermal ablation of the tumor, tumors were collected and divided into small pieces and homogenized in cold staining buffer to form single cell suspensions. Cells were stained with fluorescence-labeled antibodies CD45 (Biolegend, cat. no. 103108, Clone: 30-F11), CD11c (Biolegend, cat. no. 117310, Clone: N418), CD11b (Biolegend, cat. no. 101208, Clone: M1/70) according to the manufacturers' instructions. For detection of CAR T cells labelled with GFP in the tumor, the cells in the suspension were stained with CD4 (Biolegend, cat. no. 344614, Clone: SK3), CD8 (Biolegend, cat. no. 344706, Clone: SK1) according to the manufacturers' instructions. The stained cells were measured on a CytoFLEX flow cytometer (Beckman) and analyzed by the FlowJo software package (version 10.0.7; TreeStar, USA, 2014).

g) Statistical Analysis.

All results are presented as the mean±standard error of the mean (s.e.m.) as indicated. Tukey post-hoc tests and One-way analysis of variance (ANOVA) were used for multiple comparisons and two-tailed Student's t-test was used for two group comparison. Survival benefit was determined using a log-rank test. All statistical analyses were carried out by the Prism software package (PRISM 5.0; GraphPad Software, USA, 2007).

### V. REFERENCES

C. Feig, J. O. Jones, M. Kraman, R. J. Wells, A. Deonarine, D. S. Chan, C. M. Connell, E. W. Roberts, Q. Zhao, O. L. Caballero, Proc. Natl. Acad. Sci. U.S.A. 2013, 110, 20212-20217; bM. V. Maus, A. R. Haas, G. L. Beatty, S. M. Albelda, B. L. Levine, X. Liu, Y. Zhao, M. Kalos, C. H. June, Cancer Immunol. Res. 2013; cE. K. Moon, L.-C. Wang, D. V. Dolfi, C. B. Wilson, R. Ranganathan, J. Sun, V. Kapoor, J. Scholler, E. Puré, M. C. Milone, Clin. Cancer Res. 2014; dK. Newick, S. O'Brien, E. Moon, S. M. Albelda, Annu. Rev. Med. 2017, 68, 139-152.
C. R. Parish, Nat. Rev. Immunol. 2006, 6, 633; bW. A. Muller, Trends Immunol. 2003, 24, 326-333; cP. S. Adusumilli, L. Cherkassky, J. Villena-Vargas, C. Colovos, E. Servais, J. Plotkin, D. R. Jones, M. Sadelain, Sci. Transl. Med. 2014, 6, 261ra151-261ra151.
C. S. Hinrichs, S. A. Rosenberg, Immunol. Rev. 2014, 257, 56-71; bB. Philip, E. Kokalaki, L. Mekkaoui, S. Thomas, K. Straathof, B. Flutter, V. Marin, T. Marafioti, R. Chakraverty, D. Linch, Blood 2014, blood-2014-2001-545020; cC. S. Hinrichs, N. P. Restifo, Nat. Biotechnol. 2013, 31, 999.
G. Pluschke, M. Vanek, A. Evans, T. Dittmar, P. Schmid, P. Itin, E. J. Filardo, R. A. Reisfeld, Proc. Natl. Acad. Sci. U.S.A. 1996, 93, 9710-9715; bC. Geldres, B. Savoldo, V. Hoyos, I. Caruana, M. Zhang, E. Yvon, M. Del Vecchio, C. J. Creighton, M. Ittmann, S. Ferrone, Clin. Cancer Res. 2014, 20, 962-971; cX. Wang, A. Katayama, Y. Wang, L. Yu, E. Favoino, K. Sakakura, A. Favole, T. Tsuchikawa, S. Silver, S. C. Watkins, Cancer Res. 2011, canres. 1134.2010; dA. Morello, M. Sadelain, P. S. Adusumilli, Cancer Discov. 2016, 6, 133-146; eZ. Rivera, S. Ferrone, X. Wang, S. Jube, H. Yang, H. I. Pass, S. Kanodia, G. Gaudino, M. Carbone, Clin. Cancer Res. 2012, clincanres. 0628.2012.
G. Song, C. Liang, H. Gong, M. Li, X. Zheng, L. Cheng, K. Yang, X. Jiang, Z. Liu, Adv. Mater. 2015, 27, 6110-6117; bL. Cheng, C. Yuan, S. Shen, X. Yi, H. Gong, K. Yang, Z. Liu, ACS Nano 2015, 9, 11090-11101; cS. Stapleton, M. Dunne, M. Milosevic, C. W. Tran, M. J. Gold, A. Vedadi, T. D. Mckee, P. S. Ohashi, C. Allen, D. A. Jaffray, ACS Nano 2018, 12, 7583-7600.
H. K. Makadia, S. J. Siegel, Polymers 2011, 3, 1377-1397.
I. Caruana, B. Savoldo, V. Hoyos, G. Weber, H. Liu, E. S. Kim, M. M. Ittmann, D. Marchetti, G. Dotti, Nat. Med. 2015, 21, 524; bR. K. Jain, T. Stylianopoulos, Nat. Rev. Clin. Oncol. 2010, 7, 653; cT. Stylianopoulos, J. D. Martin, V. P. Chauhan, S. R. Jain, B. Diop-Frimpong, N. Bardeesy, B. L. Smith, C. R. Ferrone, F. J. Hornicek, Y. Boucher, Proc. Natl. Acad. Sci. U.S.A. 2012, 201213353.
J. N. Kochenderfer, M. E. Dudley, S. A. Feldman, W. H. Wilson, D. E. Spaner, I. Maric, M. Stetler-Stevenson, G. Q. Phan, M. S. Hughes, R. M. Sherry, Blood 2012, 119, 2709-2720; bB. Savoldo, C. A. Ramos, E. Liu, M. P. Mims, M. J. Keating, G. Carrum, R. T. Kamble, C. M. Bollard, A. P. Gee, Z. Mei, J. Clin. Invest. 2011, 121, 1822-1826; cR. J. Brentjens, I. Rivière, J. H. Park, M. L. Davila, X. Wang, J. Stefanski, C. Taylor, R. Yeh, S. Bartido, O. Borquez-Ojeda, Blood 2011, blood-2011-2004-348540.
K. Yang, S. Zhang, G. Zhang, X. Sun, S.-T. Lee, Z. Liu, Nano Lett. 2010, 10, 3318-3323; bQ. Chen, C. Wang, Z. Zhan, W. He, Z. Cheng, Y. Li, Z. Liu, Biomaterials 2014, 35, 8206-8214.
Q. Chen, C. Liang, C. Wang, Z. Liu, Adv. Mater 2015, 27, 903-910; bQ. Chen, H. Ke, Z. Dai, Z. Liu, Biomaterials 2015, 73, 214-230; cL. Cheng, C. Wang, L. Feng, K. Yang, Z. Liu, Chem. Rev. 2014, 114, 10869-10939; dJ. T. Robinson, S. M. Tabakman, Y. Liang, H. Wang, H. Sanchez Casalongue, D. Vinh, H. Dai, J. Am. Chem. Soc. 2011, 133, 6825-6831.
Q. Chen, L. Xu, C. Liang, C. Wang, R. Peng, Z. Liu, Nat. Commun. 2016, 7, 13193.
S. L. Maude, T. W. Laetsch, J. Buechner, S. Rives, M. Boyer, H. Bittencourt, P. Bader, M. R. Verneris, H. E. Stefanski, G. D. Myers, New Engl. J. Med. 2018, 378, 439-448; bS. S. Neelapu, F. L. Locke, N. L. Bartlett, L. J. Lekakis, D. B. Miklos, C. A. Jacobson, I. Braunschweig, O. O. Oluwole, T. Siddiqi, Y. Lin, New Engl. J. Med. 2017, 377, 2531-2544; cM. L. Davila, I. Riviere, X. Wang, S. Bartido, J. Park, K. Curran, S. S. Chung, J. Stefanski, O. Borquez-Ojeda, M. Olszewska, Sci. Transl. Med. 2014, 6, 224ra225-224ra225.

1. An engineered particle comprising a photosensitizer.

2. The engineered particle of claim 1, wherein the photosensitizer comprises a near-infrared (NIR) dye.

3. The engineered particle of claim 1, wherein the particle comprises poly(lactic-co-glycolic) acid.

4. The engineered particle of claim 1, wherein the photosensitizer is encapsulated in the engineered particle.

5. A pharmaceutical composition comprising the engineered particle of claim 1.

6. A method of treating cancer comprising administering to a subject in need thereof tumor-specific T cell population and an effective amount of the engineered particle of claim 1; and stimulating the engineered particle with light comprising a wavelength at which photosensitizer is excited.

7. A method of treating a cancer comprising administering to a subject in need thereof an effective amount of a tumor-specific T cell population and an engineered particle comprising a photosensitizer; and stimulating the engineered particle with light comprising a wavelength at which photosensitizer is excited.

8. The method of claim 6, wherein the cancer comprises skin cancer, prostate cancer, lung cancer, breast cancer, pancreatic cancer, colon cancer, gastric cancer, bladder cancer, head and neck cancer, oral cancer, cholangiocarcinoma, ovarian cancer, cervical cancer, or esophageal cancer.

9. (canceled)

10. The method of claim 1, wherein the subject is a human.

11. The method of claim 6, wherein the engineered particles are administered to the subject at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, or once every 2, 3, 4, 5, 6 months.

12. The method of claim 6, wherein 1, 2, 3, 4, 5, 6, 7, 8, 9, or 10 doses of the engineered particles are administered to the subject.

13. The method of claim 6, wherein the dose of the administered engineered particles is from about 10 mg/kg to about 100 mg/kg.

14. The method of claim 6, wherein the administering comprises intratumoral injection.

15. The method of claim 6, wherein the tumor-specific T cell population comprises CAR T, tumor infiltrating lymphocytes (TIL), effector T cell, memory T cell, effector memory RA T cell (TEMRA), or stem cell-like memory T cell.

16. The method of claim 6, wherein the light comprises a NIR light.

17. The method of claim 16, wherein the NIR light comprises a wavelength of about 650 nm to about 1000 nm.

18. The method of claim 17, wherein the duration of stimulation is from 1 min to 60 min.

19. The method of claim 6, further comprising administering to the subject at least one anti-cancer therapeutic agent.

20. The method of claim 19, wherein the at least one anti-cancer therapeutic agent comprises an immune checkpoint blockade.

21. The method of claim 20, wherein is the immune checkpoint blockade comprises an antibody targeting PD-1, PD-L1, PD-L2, or CTLA-4.
